(19) United States
(12) Patent Application Publication
(10) Pub. No.: US 2015/0105837 A1
(43) Pub. Date: Apr. 16, 2015

(54) BRAIN THERAPY SYSTEM AND METHOD USING NONINVASIVE BRAIN STIMULATION

(71) Applicant: NEUROMETRICS, S.L., Murcia (ES)

(72) Inventor: Moises AGUILAR DOMINGO, ESPINARDO (MURCIA) (ES)

(73) Assignee: NEUROMETRICS, S.L., Murcia (ES)

(21) Appl. No.: 14/055,145

(22) Filed: Oct. 16, 2013

(51) Int. Cl. A61N 1/36

(52) U.S. Cl. CPC A61N 1/36025
USPC 607/45

(57) ABSTRACT

A brain therapy system and method using noninvasive brain stimulation, comprising:
a stimuli generator unit for generating a stimuli presentation program;
a sensor assembly comprising a plurality of active EEG electrodes for measuring the electrophysiological activity of a patient's brain subjected to said stimuli presentation program;
a data acquisition unit in communication with the sensor assembly for retrieving patient EEG signals;
a data processing unit in communication with the data acquisition unit for:
   analyzing the patient EEG signals to obtain patient EEG data, and
   comparing the patient EEG data against a normalized EEG pattern retrieved from a standardized database;
   selecting a stimulation protocol in dependence of said comparison;
a brain stimulation unit for applying the selected stimulation protocol on the active EEG electrodes.

### FIELD OF THE INVENTION

The present invention pertains to the field of electroencephalography. It particularly relates to a brain therapy system and method using noninvasive brain electro-stimulation.

### BACKGROUND OF THE INVENTION

Mental disorders are common. More than one out of three people in most countries report sufficient criteria for considering at least one mental disorder at some point in their life. In the United States 46% of the population qualifies for a mental illness at some point. An ongoing survey indicates that anxiety disorders are the most common, followed by mood disorders, while substance disorders and impulse-control disorders are consistently less prevalent. A 2005 review of surveys in 16 European countries found that 27% of adult Europeans are affected by at least one mental disorder in a 12-month period.

Approximately 7% of a preschool pediatric sample was given a psychiatric diagnosis in one clinical study, and approximately 10% of 1- and 2-year-olds receiving developmental screening have been assessed as having significant emotional/behavioral problems based on parent and pediatrician reports.

The causes remain unknown, but are probably multiple. There is no cure and still no effective treatment. In addition to having a brain disease, people with serious mental illness are (by definition) significantly functionally impaired by the illness for an indefinite period of time (diagnosis, disability, duration). The problems of victims and their families are compounded by stigma.

A major management option for many mental disorders is psychotherapy. The second step is medication. There can be problems with adverse effects of medication and adherence to them, the people take long time (months, years) and not involve patient healing.

Transcranial direct current stimulation (tDCS) and transcranial alternating current stimulation (tACS) employ a small direct current that is delivered to the scalp, with different frequencies and offsets in the case of tACS, penetrating the skin, skull and meninges to reach the brain. These currents have been shown to modify neural membrane electrical potentials, leading to transient alteration of neuronal excitability and firing rates. Depending on the use of anodal or cathodal stimulation, firing rates of underlying cortical neurons are either increased or decreased, leading to transient demonstrable changes in perception or behavior. The advantages of Noninvasive Brain Stimulation are manifold, and include tolerability, ease of use, portability, and low risk of adverse events, including seizures. Given its advantages, Noninvasive Brain Stimulation has been gaining popularity both as a tool for neuroscience research and as a potential modality for the assessment and treatment of a variety of conditions, including neuropsychiatric disorders, Parkinsonism, stroke, and chronic neuropathic pain.

Actually, 80% of the devices sold are intended for neurotherapy by neurofeedback or training brainwaves, whereas only 20% are intended for tDCS or tACS.

For example, Othmer NeuroAmp brand electroencephalography (EEG) amplifier was used with a general purpose computer as programmed intended to train down average delta wave amplitude when the amplified feedback is provided through a micro coded J&J I-330 C2 EEG.

Another example is BrainMaster Atlantis 2×2 Model EEG amplifiers, used to acquire the EEG. It was programmed within the computer according to decreased average amplitude of the Delta frequency band, rather than microprogrammed on an onboard microprocessor inside the EEG amplifier.

The Low Energy Neurofeedback System (LENS) employs extremely weak intensities of feedback and does involve the patient's own EEG driving the feedback, but does not involve any conscious participation or even positive intention. The EEG electrodes serve as bi-directional conduits for both the brainwaves and the feedback signals.

The BrainStim Stimulator from E.M.S. has been developed and validated as a technique to enhance neurorehabilitation. Transcranial stimulation with low-intensity direct current (tDCS) or alternating current (tACS, tRNS) generates neuromodulations in spontaneous neuronal activity. The BrainStim Stimulator is programmed by non-dedicated computer software package. It communicates with the control computer via an integrated Bluetooth wireless interface. The appropriate therapeutic protocol is created in the computer software and saved on the BrainStim unit ready for use on a patient or subject. The simple and intuitive controls make BrainStim comfortable and ideal for use in the home setting. All data and information recorded during therapy sessions is logged and a unique digital “diary” for each experimental subject is created and maintained. Information thus created can be read directly on the computer control unit in the BrainStim software. The Stimulator device is completely controlled by microprocessor and permits the clinician to use unidirectional and bidirectional current, waveforms pulse, continuous DC, semi-sine, sine and noise with selectable band.

The tDCS device from Soterix Medical Inc., like BrainStim unit, works with 1 or 2 leads, which only deliver weak current, but not records and analyses brainwaves like Neuro-replacement solution. The Soterix Medical tDCS Low-intensity Stimulator sends a low-level current from the positive electrode, anode, to the negative electrode, cathode. Soterix tDCS apparatus is an investigational device, like Chattanooga tDCS device.

During tDCS session with this type of Brain Stimulator Unit, a weak constant current is passed across the brain using electrodes placed on the scalp; prolonged passage of current (e.g. >10 min) can lead to lasting changes in neuronal excitability. Most commonly, conductive rubber pads wrapped in saline-soaked sponge pockets are used as tDCS electrodes. Stimulation protocols for tDCS consist of a fade-in phase in which current is ramped up to the desired intensity (typically <30 s), the main stimulation phase at target intensity (typically 1-2.5 mA, for 10-20 minutes), and a fade-out phase. The voltage needed to ramp up current and maintain stimulation depends on the impedance across the body and the electrodes. Skin (scalp), skull, CSF, and brain tissue contribute to body impedance, with skin impedance known to change depending on current intensity and density and stimulation duration. Electrode impedance is a function of dynamic electrochemical processes and is also a complex function of stimulation waveform and time. It is precisely because tissue and electrode impedance across subjects and time is highly variable, that current controlled stimulation is used to ensure reproducible delivery of stimulation dose to the brain. During tDCS, the voltage is therefore adjusted to maintain the desired current level across variable impedances. Poor electrode design and preparation can thus lead to higher voltages being applied. Although current/charge density and total delivered charge are considered the main parameters causing tissue damage and painful sensation, unnecessarily high voltages are also undesirable for several reasons. Joule heating leading to temperature increase is a function of both current and voltage, though is likely not significant during conventional tDCS or High-Definition tDCS.

The Soterix Medical Brain Stimulator uses 4th order linear circuit model that decreases impedance produced by the passage of current itself significantly and reduces the compliance voltage required. Furthermore, recognizing that reaching and maintaining the target current intensity does not require a strictly controlled (linear) ramp. Soterix developers use a “Limited Total Energy” (LTE) approach to tDCS which allows robust extra-low voltage stimulation. This approach can combine: (1) prestimulation impedance thresholds (which indicate set-up conditions); (2) statistical methods; (3) Limited Total Energy (LTE) and (4) linear models and feed-back control. With these approaches Soterix Unit can increase the robustness and safety of tDCS.

Finally, Starstim is a Neuroelectrics brain stimulator helmet system that allows to stimulate using up to 8 electrodes with independent current-control setup of each electrode, allowing flexible electrode placement. With this type of electronic device independent current control of each electrode with arbitrary linear combinations of tDCS, tACS, tRNS or Sham protocol can be achieved, that is, an independent setup of each electrode-sponge/saline or small area Ag/AgCl/gel electrodes can be used, but like other apparatus from DC current application in scalp are investigational devices, with complicate management for clinicians and researchers.

Multi-electrode tDCS (MtDCS) can be achieved with Starstim stimulator, which is able to control the current in several electrodes independently. It has several advantages over traditional bipolar tDCS. One way to use an MtDCS system is to place one small electrode over the target area and several small return electrodes in distant parts of the scalp. The main effect is to decrease the electric field in the cortex under the return electrodes since only a fraction of the total current is passing through each one of them. Also, by using smaller electrodes, the electric field in the target region will be more focal. The advantages of this type of assembly are: a more focal stimulation in the target area and minimal stimulation in the rest of the cortex, thereby achieving quasi-monopolar stimulation. Another advantage of this multi-electrode assembly is the ability to exert some control over the electric field distribution and the direction of the current in the brain by altering the fraction of the current that is extracted through each return electrode.

Starstim brain stimulator helmet unit is a high definition tDCS device that can have multiple cathodes and/or multiple anodes. An array of 4 small anodes splitting 2 mA, for example (0.5 mA each electrode), can function as an anodal ‘virtual pad’ and assumes cathode somewhere else on the body.

We can assume that tACS is a form of tCS where the transchranial stimulation currents are time dependent with a sinusoidal shape (as in AC current). Amplitude, frequency and relative phases across stimulation electrodes can be controlled. tACS stimulation may provide a powerful way to couple to the oscillatory behavior of the brain.

With the brain stimulator cap tRNS (Transcranial Random Noise Stimulation) can be applied, i.e. a random electrical oscillation spectrum over the cortex. tRNS can be applied at different frequency band ranges over the entire spectrum from 0.1 to 640 Hz. After applying tRNS to the visual cortices of healthy subjects, a significant improvement in the performance of healthy subjects in a visual perceptual learning task was observed. This improvement was significantly higher than the improvement obtained with anodal tDCS.

The advantages of MtDCS over tDCS also apply to MtACS or tRNS. Different targets can be stimulated at the same frequency or at different frequencies. In Starstim system, amplitude, frequency and phase of the sine-wave can be controlled independently for each electrode.

However, none of the above mentioned inventions take into account the peculiarities of people with neurological or mental health problems and at no point comparing EEG brainwaves recorded in the sensors with patterns extracted from normal healthy population or standardized databases.

In most known devices it is quite common to use expert opinion when applying a neuromodulation protocol or references which proposes a treatment protocol based on the experience gained after treatment of a small group of patients with a particular disease or disorder, after objectify a small improvement in clinical scales or based on the professional experience of a group of experts.

### SUMMARY OF THE INVENTION

It is herewith proposed a treatment protocol using noninvasive brain electro-stimulation based on the feedback received and their deviations with respect to a normalized database, that solves the aforementioned drawbacks.

In accordance with a first aspect of the present invention there is provided a brain therapy method using noninvasive brain stimulation, comprising:
a stimuli generator unit for generating a stimuli presentation program;
a sensor assembly comprising a plurality of active EEG electrodes for measuring the electrophysiological activity of a patient's brain subjected to said stimuli presentation program;
a data acquisition unit in communication with the sensor assembly for retrieving patient EEG signals;
a data processing unit in communication with the data acquisition unit for:
   i. analyzing the patient EEG signals to obtain patient EEG data, and
   ii. comparing the patient EEG data against a normalized EEG pattern retrieved from a standardized database;
   iii. selecting an electrical stimulation protocol in dependence of said comparison;
a brain stimulation unit for applying the selected electrical stimulation protocol on the active EEG electrodes.

According to a preferred embodiment the data acquisition unit comprises at least one EEG amplifier, at least one EEG filter and a signal conditioner.

The data processing unit is preferably configured to perform an independent component analysis on the patient EEG signals.

In a yet preferred embodiment the data processing unit is configured to analyze the EEG signals to determine the dominant frequency, wavelength and average responses.

The standardized database preferably comprises EEG patterns extracted from normal healthy population. The EEG patterns from the standardized database may be structured in reference groups according to age criteria, such that the normalized EEG pattern retrieved from the standardized database corresponds to a reference group age-matched to the patient. The normalized EEG pattern retrieved from the standardized database preferably corresponds to a reference group subjected to the same or similar stimuli presentation program.

In a preferred embodiment the sensor assembly is implemented in a helmet.

The electrical stimulation protocol may comprise at least one of the following parameters:

an electrical offset value;

frequency and amplitude of the electric current waveform to apply on the active EEG electrodes;

the position of the active EEG electrodes to activate;

the duration of the stimulation process;

the number of sessions of the stimulation process.

In accordance with a further aspect of the present invention there is provided a brain therapy method using noninvasive brain stimulation, comprising:

applying a stimuli presentation program to a patient;

obtaining patient EEG signals from active EEG electrodes applied on the patient's brain;

analyzing said patient EEG signals to obtain patient EEG data;

computing the deviation between the patient EEG data and a normalized EEG pattern retrieved from a standardized database;

generating an electrical stimulation protocol based on said deviation;

applying the electrical stimulation protocol on the active EEG electrodes.

In a preferred embodiment the brain therapy method comprises measuring EEG spontaneous activity of the patient's brain and measuring EEG activity of the patient's brain in reaction to a sequence of stimuli.

The analysis of the patient EEG signals may comprise an independent component analysis, and may also comprise determining the dominant frequency, wavelength and average responses.

The brain therapy method may comprise the application of spatial averaging techniques on the patient EEG signals to obtain ERP waves and behavioral data.

The electrical stimulation protocol may comprise at least one of the following steps:

determining an electrical offset value;

selecting frequency and amplitude of the electric current waveform to apply on the active EEG electrodes;

determining the position of the active EEG electrodes to activate;

determining the duration of the stimulation process;

determining the number of sessions of the stimulation process.

### BRIEF DESCRIPTION OF THE DRAWINGS

A series of drawings which aid in better understanding the invention and which are expressly related with an embodiment of said invention, presented as a non-limiting example thereof, are very briefly described below.

FIG. 1 shows a block diagram of a brain stimulation system according to the prior art.

FIGS. 2A-2D show plots of potential brainwaves, and the resulting steady-state evoked potentials. FIG. 2A represents raw brainwaves in Fz-Cz electrodes in bipolar montage, Lemos type; FIG. 2B shows spectral analysis of 2 peaks derived by an FFT of time domain EEG; FIG. 2C shows topograms 2D of electrical EEG activity, Frontal Midline Theta at 7.08 Hz in Fz and Occipital Alpha at 10.74 Hz; and FIG. 2D shows ERP waves at Pz position.

FIG. 3 shows a block diagram of a brain computer interface and brain stimulation device according to the present invention.

FIG. 4 shows the complete methodology of the present invention.

FIG. 5 shows the time sequence of a stimuli presentation program.

### PREFERRED EMBODIMENT OF THE INVENTION

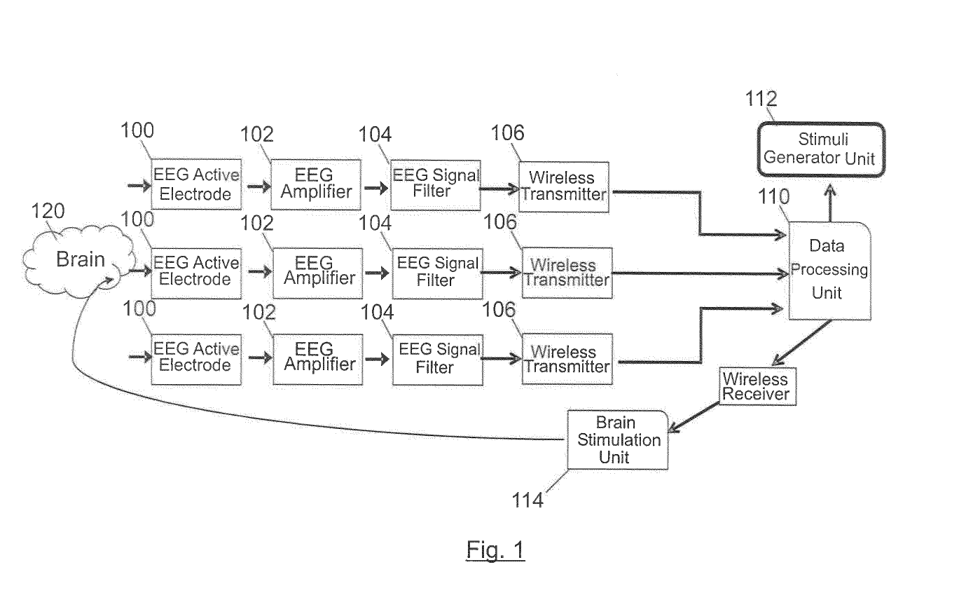

FIG. 1 shows, according to the prior art, a block diagram of a brain stimulation system, a schematic illustration of a device used to record EEG data, process electrophysiology data, and apparatus to treat cortical patches with electrical current and the supply unit of current, based on the analysis of EEG. The numerous active EEG electrodes 100 measure the electrophysiological activity of the patient's brain 120. Each EEG electrode 100 is connected to an EEG amplifier 102 and an EEG signal filter 104. Wireless transmitters 106 are configured to transmit each filtered EEG signal to a data processing unit 110, which analyzes the received EEG signals. A stimuli generator unit 112 generates different sequences of stimuli (visual, auditory, etc.), according to a program selected by the data processing unit 110. On the other hand, the data processing unit sends a stimuli signal to a brain stimulation unit 114 with batteries for generating the excitation signals applied to the patient's brain 120.

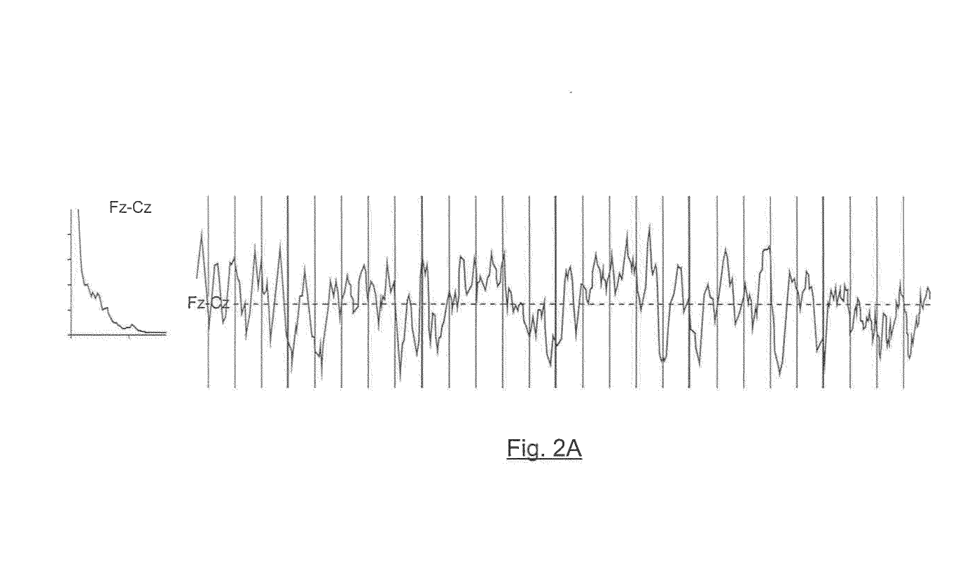

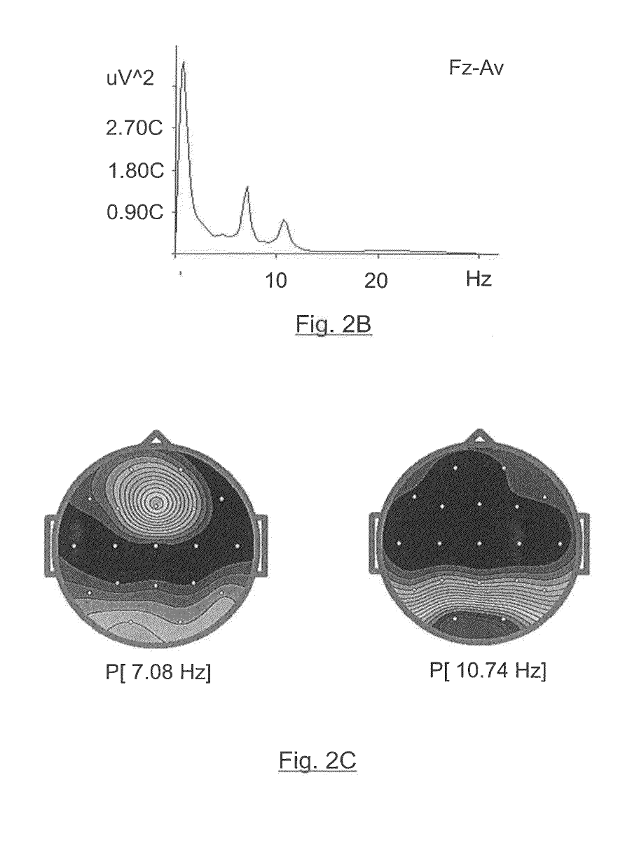

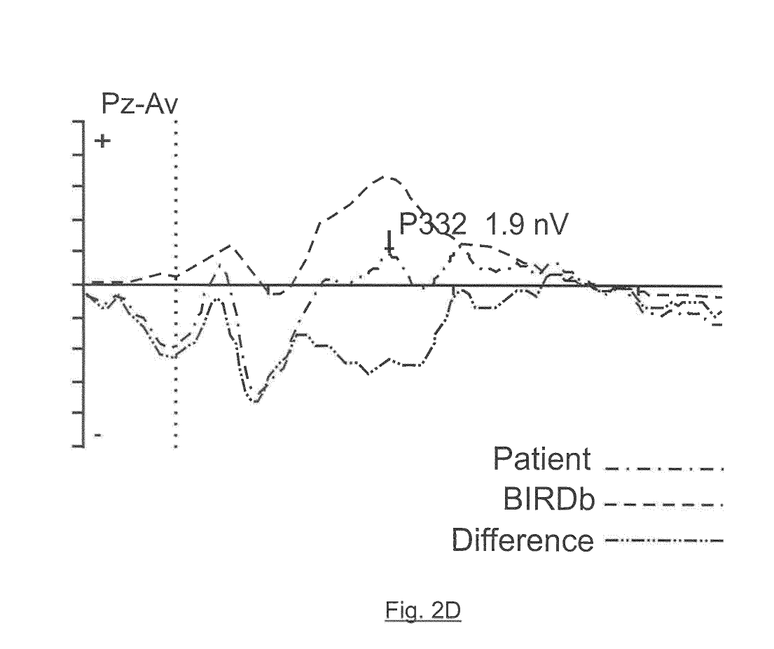

FIG. 2 represents plots of potential brainwaves and the resulting steady-state evoked potentials. The figure shows raw brainwaves in Fz-Cz electrodes in bipolar montage, Lemos type (FIG. 2A), spectral analysis of 2 peaks derived by an FFT of time domain EEG (FIG. 2B), topograms 2D of electrical EEG activity, Frontal Midline Theta at 7.08 Hz in Fz and Occipital Alpha at 10.74 Hz (FIG. 2C) and ERP waves at Pz position (FIG. 2D), with the curve of the patient, the curve from a Brainmech Index Reference Database (BIRDb), a standardized database with EEG data from normal healthy population, and the differences between both curves.

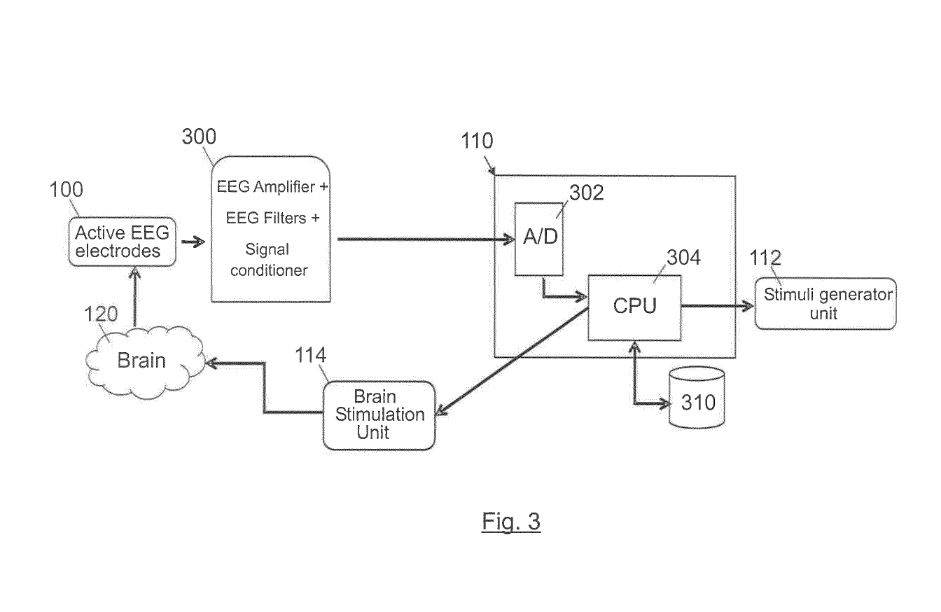

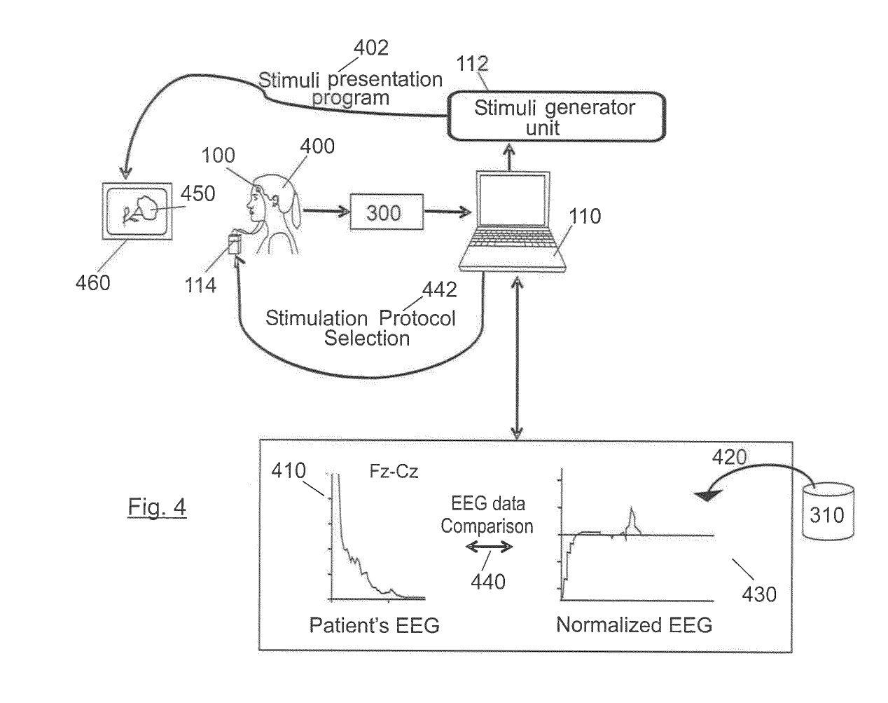

FIG. 3 shows a block diagram of a brain computer interface (BCI) and brain stimulation device according to the present invention, with a schematic illustration of the parts of the brain stimulation helmet, with EEG differential amplifier with an embedded microprocessor, sensor assembly, generator stimuli, wireless receiver and transmitter, data process unit with SVC classifier & BIRDb and brain stimulation unit with batteries.

The present invention relates to a device and method of classification and brain therapy using brain noninvasive stimulation. It is a brain computer interface (BCI) comprising a brain stimulation unit 114, active EEG electrodes 100, Quantitative EEG (QEEG) amplifiers. The electronic system based in BCI concept includes the measurement of human brain electrophysiological spontaneous activity and evoked activity after application of different stimulus by the stimuli generator unit 112. Such electrophysiological activity is measured by a data acquisition unit 300 using known EEG measurement techniques, said unit comprising EEG amplifiers 102, EEG signal filters 104 and signal conditioner. EEG device is being equipped with advanced functions, such as active EEG electrodes, differential amplifier to which the electrode is connected, filtering and gain steps, batteries, components to transmitting wirelessly or components to recording data for a subsequent transmission to computer. In a preferred embodiment the active EEG electrodes 100 are arranged in a cap or helmet. The data acquisition unit 300 may, for instance, be placed in a casing fixed to the helmet, and powered by a supply energy unit (one or more batteries).

The measured electrophysiological brain activity signal is an electroencephalogram (EEG) signal. The early Event Related Potentials (ERP) components comprise an EEG signal related to perception, such as the N170 wave (MMN) or mismatch negativity signal. The P300 wave is related to attention. The control software of BCI system use EEG signal analysis, such independent component analysis (ICA), wavelet transformation, or pre-processing spatial filters or automatic artifact rejection functions.

The expert system unit after presentation of multiple sequences of stimuli uses them to determine the predefined electrophysiological signal from a number of electrophysiological activity measurements. Measured data of spontaneous and evoked electrical potentials can be compared with data from a standardized database 310 with patterns extracted from normal healthy population (Brainmech Index Reference Database (BIRDb)). By comparing the data by means of parametric statistical procedures the differences between the patients and their appropriate age-matched reference group can be calculated (FIG. 2D shows the difference graph, for the ERP wave at Pz position, between the patient and the reference group in the standardized database). This computer analysis then serves as a tool in the aid of neuro-modulation planning approach.

No prior BCI system has made the specific step of using online EEG signals, related to an early electrophysiological associated or not with perceptual stimuli, to activate or deactivate different brain areas after direct current or alternate direct current application under active electrode. With this type of brain stimulation we can change plasticity of cortical area under electrode or improve the connectivity related with the flow of information with adjacent areas.

FIG. 4 shows a flow chart of the invention treatment and method process that deploys the BCI neurometrics system according to the present invention. The invention is a new methodology for studying dynamically the interaction between different brain networks during sensory, memory, executive and emotional processes, and more particularly, to use brain noninvasive stimulation like direct current, alternate current at different frequencies or random noise to change in real-time the type of this interactions between cortical networks. After recording EEG signals, they are compared against a standardized database 310, considering behavior parameters like reaction time, omission errors, and commission errors during visual CPT Task, EEG spectral data after applied FFT to every EEG signal received from sensors. Current density of every structure of human brain, obtained from ERP signal, is compared against the standardized database 310. After this complex calculations, we know defective connectivity brain-mapping, in relation with alterations in flow of information and interaction between neural networks.

A method to change plasticity of any cortical brain area, comprises:
1.—Measuring EEG spontaneous activity.
2.—Measuring EEG activity in reaction to a sequence of stimuli generated by the stimuli generator unit 114.
3.—Matching the measured EEG activity in both conditions with a predefined EEG signatures or normalized EEG patterns (independent components).
4.—Matching every EEG signature with cognitive process. Modulation of information flow in each of the systems is characterized by specific rhythms while stages of information processing in these systems are reflected in specific Event Related Potential (ERP) components.
5.—Any of the brain systems obeys to the inverted U law:
   The law claims that responses of the system are largest if the activity of the system stays within the normal range and are abnormally smaller if the overall level of activation of the system is below or higher than the normal range.
   The level of activation of the system can be assessed by spectral analysis of the spontaneous EEG generated by the cortical part of the system. In general, excess of alpha activity in comparison to the normative range is associated with hypo-activation of the system, while excess of beta activity is associated with hyper-activation of the system.
   The responses of the system are reflected in amplitude and latency of the components generated by cortical areas of the system.
6.—Constructing neuro-modulation protocol on the basis of quantitative electroencephalogram (QEEG & ERP) assessment. After computation differences with standardized database 310 we provide brain noninvasive stimulation protocol when a match is detected on IC cognitive process impairment. A tremendous amount of empirical knowledge in EEG analysis reveals some abnormal QEEG patterns associated with various medical and psychiatric disorders. The protocols are programmed based in Bulldozer Principle. The brain state (including any dysfunction or dysregulation) is objectively reflected in parameters of EEG recorded from the scalp. According to this principle the aim of neuro-modulation is to normalize a pathologically abnormal EEG pattern. So, if there is an excess of some EEG parameter in a particular subject and in particular location in the cortex, the aim of the neuro-modulation is to train this parameter down, and if there is a lack of some other EEG characteristic the corresponding neuro-modulation parameter is trained up. The method works like a bulldozer filling in the cavities and excavating the bumps. Neurotherapy is a tool for modulating the brain on the basis of electrophysiology. Neurotherapy is the best way of activating or suppressing the impaired brain system.
7.—Launch a feedback process, wherein a new data are recorded after applied current in the human scalp, in which the difference between the first EEG signature and the second EEG signature, change in real-time the type of current, offset, time and waveform that the stimulation unit releases into the brain surface.
   The software shows maps of brain activity before, during and after stimulation. The system works according to the strength of the measured EEG signal.

As it is shown in FIG. 4, an experimental subject 400 is subjected to a sequence of stimuli 450 (for instance, an image/video shown in a display 460) provided by a stimuli generator unit 112. An expert first selects an appropriate stimuli presentation program 402 (visual, auditory, etc.) in the data processing unit 110, which controls the stimuli generator unit 112 (although in other embodiments the stimuli generator unit 112 may be autonomously controlled, or controlled by another entity). The stimuli presentation program is executed to generate certain responses. The neurological response of the experimental subject is measured using a sensor assembly (data acquisition unit 300 with differential EEG amplifiers). The data obtained by active EEG electrodes 100 positioned over scalp of experimental subject 400 are recorded. These EEG measurements are processed in a data processing unit 110, obtaining appropriate result data (patient's EEG 410) to compare 440 against the standardized database 310 and to generate an output to the brain stimulation unit 114. To that end, the data processing unit 110 accesses the standardized database 310 (which may be part of the data processing unit 110 or an external database) to retrieve 420 normalized EEG data 430 corresponding to an appropriate reference group, normally an age-matched healthy group subjected to the same (or a similar) stimulation program. The data processing unit 110 compares 440 the normalized EEG data 430 with the patient's EEG data 410, and selects 442 a stimulation protocol based on said comparison, generating the appropriate electrical stimulation signals. The stimulation protocol selected may consider the region/s of the scalp to stimulate (particular active EEG electrodes 100 chosen as anodes and cathodes), type of electrical current (DC or AC), frequency and amplitude of the current, electrical offset, duration of the stimulation and number of sessions. The chosen stimulation protocol may be predefined according to the pathology detected. The electrical stimulation signals generated are then transmitted to the brain stimulation unit 114, which applies them on the active EEG electrodes 100. Hence, the data processing unit 110 controls the brain stimulation unit 114, which is connected to the sensor assembly, which in turn provides feedback to the data processing unit 110.

The stimuli generator unit 112 can provide different sensory stimuli: visual (on a monitor screen), gustatory (using dispensatory system), olfactory (using dispensatory) and auditory (using sound card and speakers).

Stimuli generator unit 112 is controlled by the data processing unit 110, using a generator stimuli unit program. Data processing unit 110 also is in charge of EEG acquisition, using an EEG recorder program. EEG recorder program sends synchronization codes to the stimuli generator unit program to control its work and provides a synchronous stimuli presentation with EEG acquisition. The recorded data can be used to calculate and analyze event related potentials (ERP) and/or event related desynchronization (ERD). The data processing unit 110 measures the reaction time and task performance parameters (omission and commission errors) and save them to build-in database for the future analysis and final report preparation. To measure the reaction time, the user may use keyboard and mouse.

In a preferred embodiment each stimuli presentation program 402 is stored in a text (ASCII) file. It includes a list of stimuli, list of trials, list of presentation commands (the sequence of trials in simplest case) and list of response processing commands. The list of stimuli should include all files used in stimuli presentation program, such as:
All picture and sound files presented in the task.
All picture files displayed during inter-stimuli time intervals (so named background pictures).
All picture files used as description of subject task.

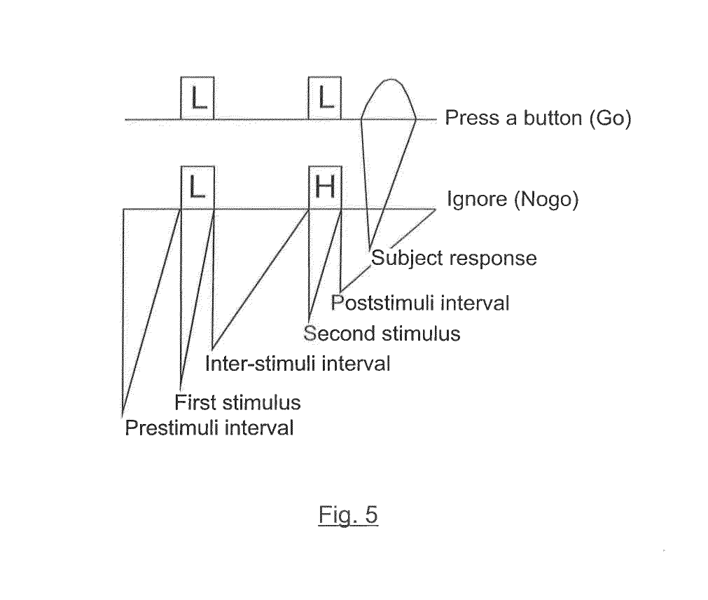

Each stimulus is described by its type (sound, image, tastes, flavors, text or impulse), name (text identifier) and additional parameter as name of corresponding file or index of textual stimulus stored in list of textual stimuli. FIG. 5 below shows schematically the time sequence of stimuli presentation in this task. The patient's reaction time may be measured using a keyboard (“press a button”).

Back to FIG. 3, the data acquisition unit 300 sends the analog EEG signals to the data processing unit 110, which has an A/D converter 302 and a CPU 304. The sample frequency is between 250 and 500 Hz. The EEG amplifier collects the relatively weak EEG signals via the active electrodes and amplifies them sufficiently for analysis by the data processing unit 110 with very fast CPU 304. The data processing unit 110 has the means to perform a Fast Fourier Transform (FFT) to converter the time domain EEG to a frequency domain resulting in a power spectrum.

When a brief stimulus 450 is presented to a patient, there is a transient brain response due to that stimulation. In EEG waves appears a small periodic signal at the same frequency as the stimulation, and harmonics may be present. Therefore the stimulus 450 interacts with intrinsic rhythms. Then, steady-state evoked potentials are produced. This evoked response is non-linear, or linear superposition of successive discrete responses, to produce a complex periodic wave. The frequency of this periodic wave can be predicted by using simple linear superposition. That transient evoked potentials exhibit correlations with attention and mental task. The speed of cortical responses is one factor that determines the frequency distribution of an EEG rhythm.

The frequency characteristics of the individual cortical responses become manifested in the power spectral density of the resulting EEG wave. Components of individual cortical responses produce lower frequencies in the composite power spectrum in endogenous EEG activity. The relationship between late ERP components and endogenous rhythms becomes clear. An endogenous rhythm consists of a train of “intrinsic evoked potentials” which are elicited by thalamocortical interaction, rather than by sensory stimulation. Evoked potentials are difficult to measure because they represent the activity only a small portion of the cells producing surface potentials, and they are mixed in the background EEG noise.

The main communication channel between the sensor assembly comprising the active EEG electrodes 100 and the data acquisition unit 300 is preferably carried out using wireless communication to eliminate signal interferences with the frequency (50 or 60 Hz) caused by the use of cables.

The batteries in the supply energy unit are connected to special electrical circuits where the reference electrode is derived to experimental subject's earlobe, neck, arm or leg to reduce common-mode-signal

Evoked potentials are generally measured by averaging techniques, and they require one or two minutes for the acquisition of a single wave. The present invention uses a new technique for measuring evoked potentials in a more rapid manner, combining repetitive stimulation with synchronous filtering, monitoring the changing of the evoked wavelets over shortened time periods, as limited by the filtering response time-constant. With this technique we can do ongoing measurement of evoked activity to show changes occurring over periods as short as 5 seconds. The data processing unit 110 performs the time-frequency decomposition of EEG signal and source time series using Morlet wavelets. The shape scaled versions of complex-valued sinusoids weighted by a Gaussian kernel of the Morlet wavelets can efficiently capture bursts of oscillatory brain activity. The temporal and spectral resolution of the decomposition is adjusted by data processing helmet unit, depending on the EEG filtered data received from the scalp of the experimental subject. EEG matrix is decomposed in the space, time, and frequency dimensions. The software of the data processing unit 110 has been efficiently designed to either store the transformed data or compute it on the fly. Data can be analyzed as instantaneous measurements, or grouped into temporal and spectral bands of interest such as alpha (8-12 Hz), theta (4-8 Hz), and so forth. Even though this reduces the resolution of the decomposition, it may benefit the analysis in multiple ways: reduced data storage requirements, improved signal-to-noise ratio, and a better control over the issue of multiple comparisons by reducing the number of concurrent hypothesis being tested.

The resultant EEG signal is then sent to a filter and filtered at a predefined frequency. Also, the data processing unit 110 subtracts muscles and eye movement's artifacts. The evoked responses can then be feedback, in real-time, to the subject. The feedback reflects the brain's responses to the repetitive stimulation, and allows the subject to receive real-time feedback regarding their attention.

After correct filtration, the data process block carefully inspects the evoked potential waveforms, and identify particular peaks and valleys (positive and negative transitions) with particular amplitudes and latencies. This features change every 4 or 5 seconds. These waveforms are comparing with the standardized database 310 to estimate variance and odds ratio from normal subjects, and after this process the system proposes or selects one stimulation protocol, wherein the result of the calculation is deemed a prospective treatment:
a) Offsets.
b) Type of electrical current: Frequency (Hertz) and amplitude (microamperes).
c) Position of electrodes, because there is active generation and delivery of weak electrical current to the patient by additional hardware and software components.
d) Duration of stimulation.
e) Number of sessions.

Recent approaches to the analysis of ERPs waves using independent component analysis and wavelet de-noising have made significant advances in reducing the problems caused by the variability of the ERP across trials for data analysis purposes. Improvements to the data process unit have been made by applying support vector machine techniques (SVM), reducing the number of trials required to reliable classification to between two and three.

Then this methods using repetitive stimulation in conjunction with brain stimulation unit over some of the active electrodes placed on a subject's scalp change the connectivity between brain networks of the experimental subject changing their plasticity. Thus, with brain stimulation neurotherapy the pathways can be changed without doing training (passive mode).

We claim:

1. A brain therapy system using noninvasive brain stimulation, comprising:
a stimuli generator unit for generating a stimuli presentation program;
a sensor assembly comprising a plurality of active EEG electrodes for measuring the electrophysiological activity of a patient's brain subjected to said stimuli presentation program;
a data acquisition unit in communication with the sensor assembly for retrieving patient EEG signals;
a data processing unit in communication with the data acquisition unit for:
analyzing the patient EEG signals to obtain patient EEG data, and
comparing the patient EEG data against a normalized EEG pattern retrieved from a standardized database;
selecting an electrical stimulation protocol in dependence of said comparison;
a brain stimulation unit for applying the selected electrical stimulation protocol on the active EEG electrodes.

2. The brain therapy system according to claim 1, wherein the data acquisition unit comprises at least one EEG amplifier, at least one EEG filter and a signal conditioner.

3. The brain therapy system according to claim 1, wherein the data processing unit is configured to perform an independent component analysis on the patient EEG signals.

4. The brain therapy system according to claim 1, wherein the data processing unit is configured to analyze the EEG signals to determine the dominant frequency, wavelength and average responses.

5. The brain therapy system according to claim 1, wherein the standardized database comprises EEG patterns extracted from normal healthy population.

6. The brain therapy system according to claim 5, wherein the EEG patterns from the standardized database are structured in reference groups according to age criteria.

7. The brain therapy system according to claim 6, wherein the normalized EEG pattern retrieved from the standardized database corresponds to a reference group age-matched to the patient.

8. The brain therapy system according to claim 7, wherein the normalized EEG pattern retrieved from the standardized database corresponds to a reference group subjected to the same stimuli presentation program.

9. The brain therapy system according to claim 1, wherein the sensor assembly is implemented in a helmet.

10. The brain therapy system according to claim 1, wherein the electrical stimulation protocol comprises at least one of the following parameters:
an electrical offset value;
frequency and amplitude of the electric current waveform to apply on the active EEG electrodes;
the position of the active EEG electrodes to activate;
the duration of the stimulation process;
the number of sessions of the stimulation process.

11. A brain therapy method using noninvasive brain stimulation, comprising:
applying a stimuli presentation program to a patient;
obtaining patient EEG signals from active EEG electrodes applied on the patient's brain;
analyzing said patient EEG signals to obtain patient EEG data;
computing the deviation between the patient EEG data and a normalized EEG pattern retrieved from a standardized database;
generating an electrical stimulation protocol based on said deviation;
applying the electrical stimulation protocol on the active EEG electrodes.

12. The brain therapy method according to claim 11, comprising measuring EEG spontaneous activity of the patient's brain and measuring EEG activity of the patient's brain in reaction to a sequence of stimuli.

13. The brain therapy method according to claim 11, wherein the analysis of the patient EEG signals comprises an independent component analysis.

14. The brain therapy method according to claim 11, wherein the analysis of the patient EEG signals comprises determining the dominant frequency, wavelength and average responses.

15. The brain therapy method according to claim 11, wherein the standardized database comprises EEG patterns extracted from normal healthy population.

16. The brain therapy method according to claim 15, wherein the EEG patterns from the standardized database are structured in reference groups according to age criteria.

17. The brain therapy method according to claim 16, wherein the normalized EEG pattern retrieved from the standardized database corresponds to a reference group age-matched to the patient.

18. The brain therapy system according to claim 17, wherein the normalized EEG pattern retrieved from the standardized database corresponds to a reference group subjected to the same stimuli presentation program.

19. The brain therapy method according to claim 11, comprising the application of spatial averaging techniques on the patient EEG signals to obtain ERP waves and behavioral data.

20. The brain therapy method according to claim 11, wherein the electrical stimulation protocol comprises at least one of the following:
determining an electrical offset value;
selecting frequency and amplitude of the electric current waveform to apply on the active EEG electrodes;
determining the position of the active EEG electrodes to activate;
determining the duration of the stimulation process;
determining the number of sessions of the stimulation process.
